(19) United States
(12) Patent Application Publication
(10) Pub. No.: US 2021/0259566 A1
(43) Pub. Date: Aug. 26, 2021

(54) METHOD AND DEVICE FOR VALIDATING A BLOOD PRESSURE MEASUREMENT SYSTEM

(71) Applicant: CNSYSTEMS MEDIZINTECHNIK GMBH, GRAZ (AT)

(72) Inventors: Jürgen Fortin, Graz (AT); Christian Fellner, Graz (AT); Julian Grond, Graz (AT); Katja Lerche, Pirching Am Traubenberg (AT); Thomas Brunner, Grambach (AT)

(21) Appl. No.: 17/255,302

(22) PCT Filed: May 12, 2020
(86) PCT No.: PCT/AT2020/060194

§ 371 Date: Dec. 22, 2020

(30) Foreign Application Priority Data
May 22, 2019 (AT) A 50469/2019

(51) Int. Cl. A61B 5/0225; A61B 5/021; A61B 5/00

(52) U.S. Cl. CPC A61B 5/02255; A61B 5/02116; A61B 2562/0238; A61B 5/6843; A61B 2560/0223; A61B 5/7221

(57) ABSTRACT

The invention relates to a method and an apparatus for validating a continuously measuring, non-invasive blood pressure measuring system equipped with a plethysmographic system capable of obtaining—in a measuring phase—a plethysmographic signal v(t) at an extremity, having a control mechanism to which the signal v(t) from the plethysmographic system is fed, which varies the contact pressure p_{C}(t) at the extremity via a control value u(t), and having an evaluation unit which continuously determines the arterial blood pressure curve p_{A}(t) based on the resulting contact pressure p_{C}(t). According to the invention, the blood pressure measuring system has a coupling interface via which—in a validation or test phase—a signal derived from a previously recorded blood pressure curve can be input into the blood pressure measuring system.

The invention relates to a method and an apparatus for validating a continuously measuring, non-invasive blood pressure measuring system equipped with a plethysmographic system which is suitable for obtaining a plethysmographic signal at a limb, which is supplied to a control mechanism which varies a contact pressure at the limb, wherein the arterial blood pressure curve is continuously determined based on the contact pressure.

The present invention describes test systems or simulators and the associated validation methods for continuously measuring, non-invasive arterial pressure monitors (Continuous Non-invasive Arterial Pressure CNAP). The measurement methods of CNAP devices are often also referred to as “Peňáz Principle”, “Vascular Unloading Technique” or “Volume Clamp Method”, although newer measurement methods described below go beyond these basic principles.

These CNAP devices detect the blood pressure signal of a living being in real time without the need to insert a catheter into the arterial vascular system. The accuracy of these CNAP devices in comparison to the actual intra-arterial blood pressure has increased enormously in the recent past due to new types of measuring sensors and new methods (algorithms) and it is to be expected that in the future they will be indispensable in daily clinical use. For acceptance in clinical use it will be necessary to check the accuracy at regular intervals. In particular, standardized test systems, simulators and validation methods are necessary for regulatory approval and subsequently for the marketing of new CNAP devices or their algorithms (methods that are mapped in software).

In CNAP systems for continuous blood pressure measurement, the blood pressure signal is recorded at one extremity (usually the finger) using a special procedure and device. The device has signal transducers for the acquisition of a so-called plethysmographic signal (volume signal) v(t) as well as means for changing the contact pressure (counterpressure) p_{C}(t) of the sensor system.

The measurement methods for these continuous CNAP systems essentially function according to the following logic: First, the contact pressure p_{Co}(t) is determined, at which the pulsations in the volume signal v(t) become maximum. According to the so-called “oscillometric principle” or “principle of maximum amplitude”, the contact pressure p_{Co}(t) at this point corresponds to the mean arterial blood pressure mBP. The search for the maximum pulsations in v(t) and thus for mBP, wherein the contact pressure p_{C}(t) is successively changed, is called “open loop phase” according to the state of the art.

If p_{Co}(t) is found, then the so-called “closed loop phase” begins. In this actual measurement phase, the CNAP system attempts to continuously maintain the oscillometric principle by means of a closed control loop. Specifically, the contact pressure p_{C}(t) of CNAP follows the natural fluctuations in actual arterial blood pressure p_{A}(t), always maximizing the amplitude of pulsations.

The known CNAP procedures now differ in how it is ensured that the system is at the point of maximum pulsation:

The method for the bloodless determination of blood pressure was presented in 1973 by Jan PENAZ (Digest of the 10th International Conference on Medical and Biological Engineering 1973 Dresden). The “Vascular Unloading Technique” or “Volume Clamp Method” is therefore also named after its inventor “Peňáz Principle”. In this method, a finger is transilluminated and a pressure is applied to the finger by means of a servo control in such a way that the originally pulsatile flow registered by the transillumination is kept constant with the aid of an electro-pneumatic control circuit. The “principle of maximum amplitude” can be maintained as long as no changes occur due to the smooth vascular muscles (vasomotor function).

This disadvantage was eliminated according to U.S. Pat. No. 4,510,940 A (WESSELIG), wherein the “closed loop phase” is regularly interrupted. In the following “open loop phase” a new short search for the maximum amplitudes according to given criteria is performed.

Both PENAZ and WESSELING use only one control loop for their method, which is to correct all disturbance variables. U.S. Pat. No. 8,114,025 B2 (FORTIN) describes a new CNAP method in which interlocking concentric control loops are each responsible for a specific disturbance variable. In U.S. Pat. No. 8,814,800 B2 (FORTIN), the “principle of maximum amplitude” is further improved; after each heartbeat the correctness of the principle is checked by using a special property of the pulse shape to correct the operating point.

In U.S. Pat. No. 10,098,554 B2 (FORTIN), a new CNAP principle is described, which differs from the simple “Volume Clamp Method”: the contact pressure p_{C}(t) no longer has a pulsatile effect on the extremity, but only follows slowly the changes of the mean arterial blood pressure mBP. The pulsatile nature of the continuous blood pressure curve is obtained from the pulsatile plethysmographic signal v(t). In WO 2016/110781, a variant of a portable blood pressure monitoring system (CNAP-2-GO) is described.

For all known systems, it is true that they only work if an extremity (e.g. the finger) of a living being is brought into contact with the measuring sensors.

It is the object of the invention to improve a method and an apparatus for validating a continuously measuring, non-invasive blood pressure measuring system in such a way that no extremity (for example a finger) needs to be brought into contact with the device for the validation of the blood pressure measuring system.

According to the invention, this is achieved by—in a validation or test phase—feeding a signal derived from a previously recorded blood pressure curve into the blood pressure measuring system via a coupling interface.

Advantageous variants of the validation method according to the invention are set out in the dependent method claims 2 to 10.

An apparatus according to the invention for the validation of a continuously measuring, non-invasive blood pressure measuring system equipped with a plethysmographic system which is suitable—in a measuring phase—for obtaining a plethysmographic signal v(t) at an extremity is characterized in that the blood pressure measuring system has a coupling interface via which—in a validation or test phase—a signal derived from a previously recorded blood pressure curve can be input into the blood pressure measuring system.

Advantageous variants of the apparatus according to the invention are set out in dependent claims 12 to 15.

The apparatus and method according to the invention are fundamentally different from known test systems used for the validation of, for example, intermittent blood pressure monitors. These intermittent blood pressure monitors usually measure the currently occurring blood pressure on the upper arm or wrist in a spot measurement without displaying the continuous curve. The cuff is inflated above the systolic blood pressure value and then the pressure is slowly released. Exactly one systolic, mean or diastolic value is obtained per inflation, which usually takes up to one minute. The evaluation procedures of intermittent blood pressure measurement methods are usually the auscultatory procedure according to Riva-Rocci (1896)/Korotkow (1905), in which the sounds in the crook of the arm are listened to with a stethoscope, or the oscillometric procedure, which is often used in automatically functioning devices.

In literature, test systems for intermittent blood pressure monitors are described in which the oscillometric pulses or “Korotkow tones” are artificially brought into the blood pressure monitor to be tested in order to enable the measurement of a given blood pressure value. These test systems cannot be used for continuously measuring CNAP systems; they cannot close the control loop necessary for these CNAP systems nor can they simulate the physiological characteristics of the extremity (e.g. fingers).

The initially described methods of the state of the art have one thing in common: in the “closed loop phase”, the closed CNAP control system attempts to keep the volume changes v(t) or, according to U.S. Pat. No. 10,098,554 B2, specific frequency contents of v(t) (referred to as v_{F}(t)) constant by changing the contact pressure p_{C}(t). As will be described in detail later, v(t) or certain frequency contents v_{F}(t) indicate how large the deviation between the actual contact pressure p_{C}(t) and the actual arterial blood pressure p_{A}(t) is, since the deviations p_{C}(t)-p_{A}(t) also result in volume changes. In the language of system and control engineering, v(t) or v_{F}(t) corresponds to the control deviation, also known as error signal e(t). If the control deviation e(t) is sufficiently small, p_{A}(t) can be reconstructed with sufficient accuracy from p_{C}(t)—of course without having to determine p_{A}(t) using an invasive catheter, which is the great advantage of these CNAP systems. With the newer methods according to U.S. Pat. No. 10,098,554 B2 and WO 2016/110781 A1, further frequency contents of v(t) are required in addition to p_{C}(t) for the determination of pulsatile p_{A}(t).

A further essential feature of all CNAP systems is the fact that the comparative element of the controlled system—i.e. the place where the “reference variable” p_{A}(t) is compared with the “controlled variable” p_{C}(t)—is located in the body or in the extremity of the living organism. From a control engineering point of view it is irrelevant where the so-called “comparator” is located. In practice, however, this means that a conventional CNAP system only works if an extremity or finger of a living being is available—i.e. even if the CNAP system is to be tested or validated.

The CNAP system therefore does not have the exact reference variable—i.e. the actual arterial blood pressure p_{A}(t)—at its disposal. It only receives the plethysmographic information, i.e. a signal corresponding to the changes in the volume v(t) of the artery. This information is usually obtained by a light procedure, in which a light source (e.g. LED) is attached to one side of the extremity (e.g. finger) and the transmitted signal is measured on the other side (e.g. using a photodiode). With all known systems, the light source emits a light with constant luminous intensity, or in most cases constant, rectangular light pulses are used. The use of light pulses allows on the one hand a higher energy density with reduced energy expenditure and on the other hand allows the detection of ambient light during the so-called blanking interval. Light systems for CNAP devices are described for example in U.S. Pat. No. 8,343,062 B2 (FORTIN).

Until now, the reference variable p_{A}(t) could only be brought into a CNAP system if an extremity (e.g. finger) of a living being was present. Logically, only the current blood pressure of the test person can be measured. For regulatory reasons, this is a major disadvantage, as it means that no standardized, repeatable test measurements can be performed. Systematic examinations of the CNAP system are thus not possible. In contrast to other biomedical systems such as the ECG or the test systems for intermittent blood pressure monitors mentioned above, it is not possible to record signals once and test them repeatedly. Problematic blood pressure curves such as blood pressure drops during operations, asystole during tilting table examinations, changes during different pacemaker settings, etc. cannot be reproduced. Changes to the CNAP system must be validated for regulatory reasons. According to the state of the art, this is only possible by repeated test measurements on the patient in the clinic. Since such problematic blood pressure curves are unpredictable, measurements on a large number of patients are necessary to obtain statistically significant results.

The present invention now enables standardized validation measurements to be carried out. In the following, several embodiment variants of apparatuses and methods for the validation of CNAP systems are described.

A basic method of feeding the previously recorded blood pressure curve as a reference variable p_{A}(t) is done by modulating the light, specifically by modulating the LED light pulses. With the apparatus according to the invention, the CNAP system can no longer distinguish whether the resulting light signal v(t) on the receiving side was generated by a simulation module of the apparatus or whether the modulation originates from a finger of a person. The CNAP system subsequently generates a contact pressure p_{C}(t) via the described control system from which the arterial blood pressure p_{A}(t) to be determined can be derived. The controlled variable p_{C}(t) is in turn fed into the simulation module and compared with the continuing recorded reference variable p_{A}(t). A new v(t) is generated and again fed into the system via modulation of the light and the control loop is closed.

This makes it possible that a clinically recorded blood pressure curve—more precisely, a patient's blood pressure signal once recorded—can be fed into the CNAP system under test. The resulting measurement signal can then be compared with the input signal. It is irrelevant whether the patient signal originates from an intra-arterial catheter or from a CNAP device used during the clinical measurement.

The invention further describes validation methods of CNAP systems which can be performed with the present test system or simulator as well as methods which can prove an equivalence of already approved CNAP systems with new, modified systems.

The invention is explained in more detail in the following by means of embodiment examples, wherein:

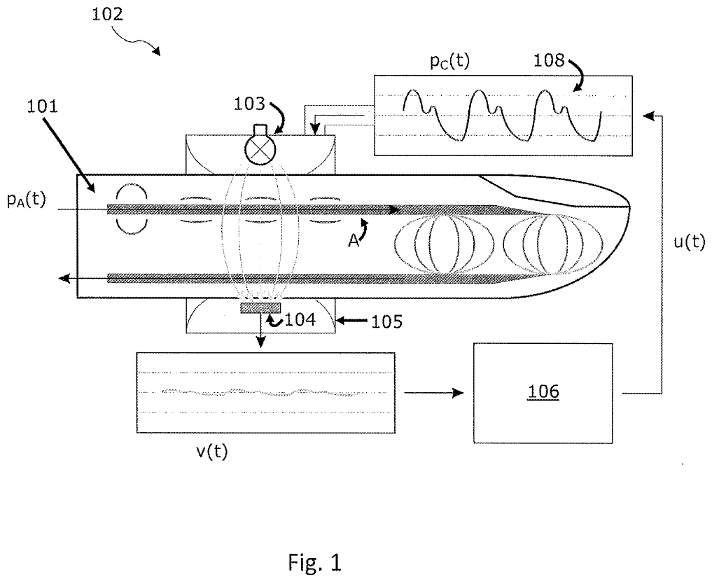

FIG. 1 shows a blood pressure measurement system in schematic illustration with a CNAP control circuit according to the state of the art in the measurement phase;

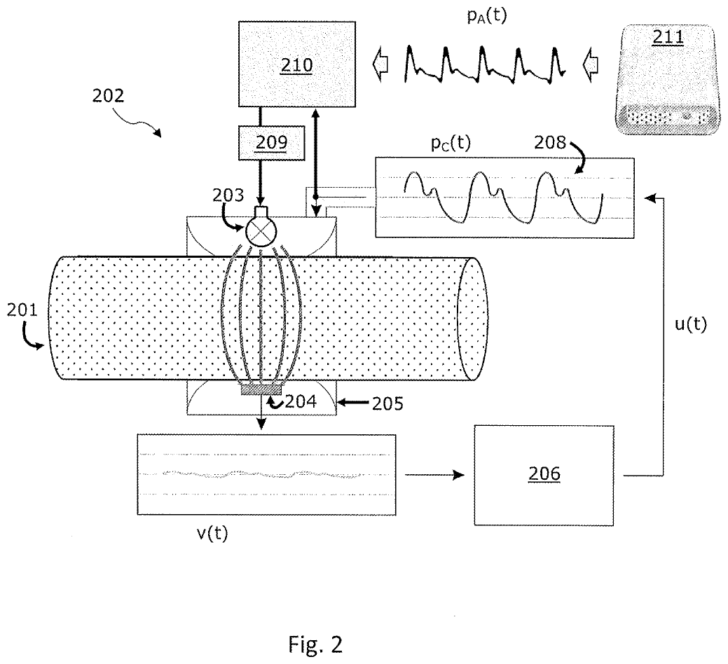

FIG. 2 shows a validation apparatus, according to the invention, with a blood pressure measuring system as shown in FIG. 1;

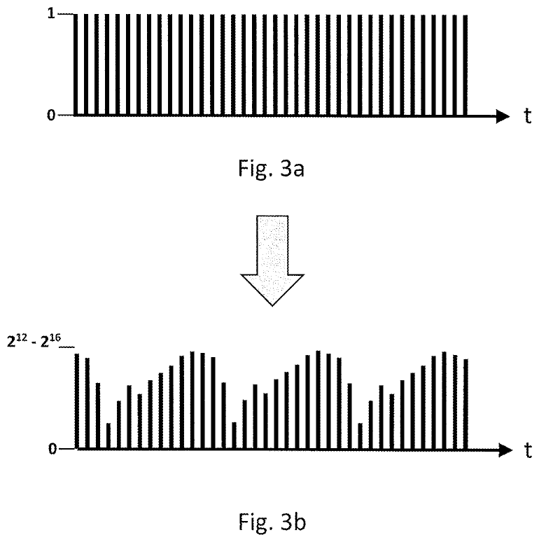

FIG. 3a and FIG. 3b show diagrams of LED pulses;

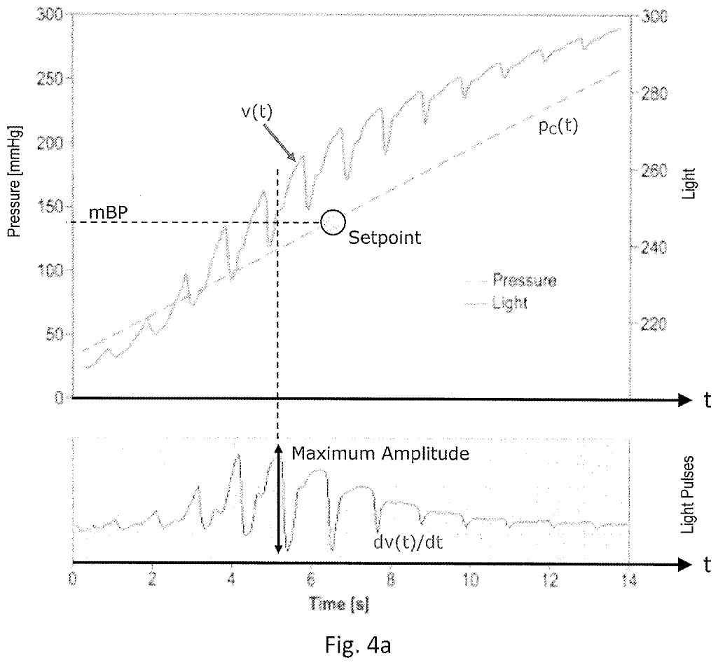

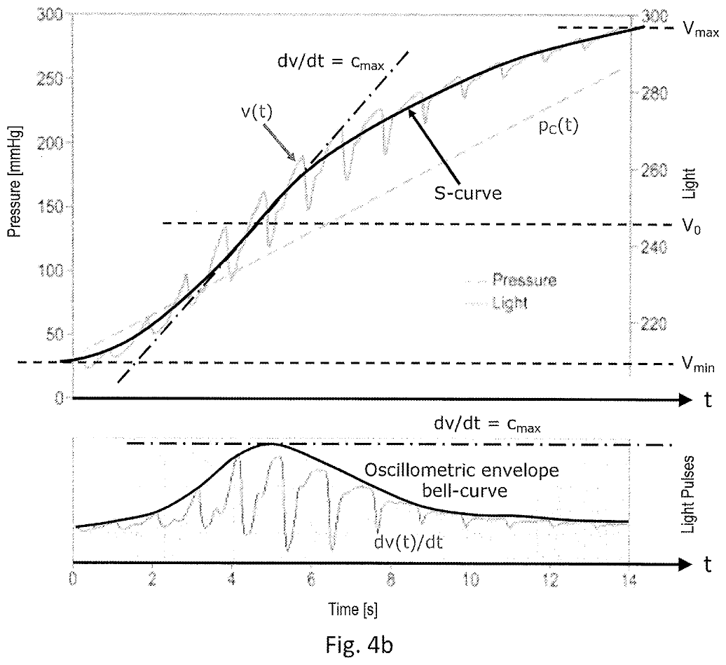

FIGS. 4a and 4b show p-v diagrams to determine the setpoint; and

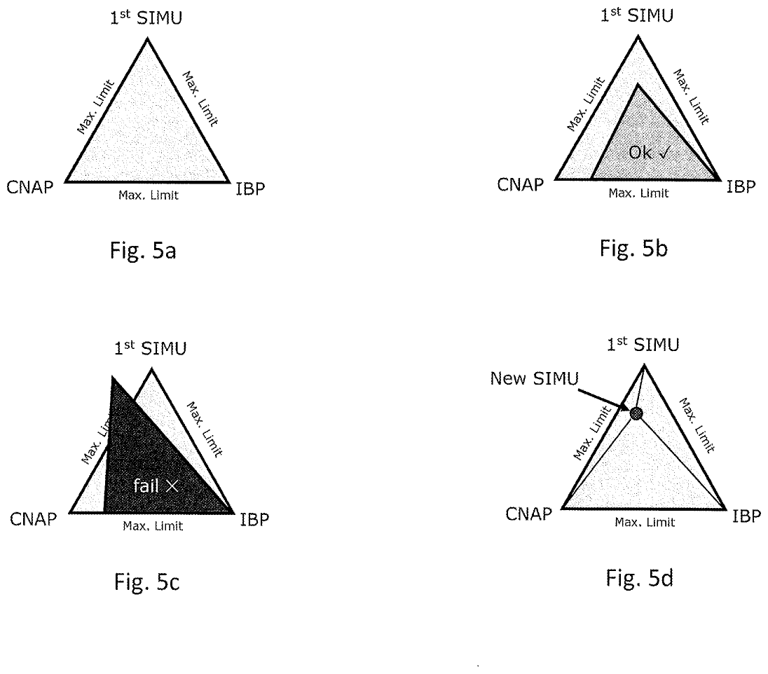

FIGS. 5a to 5d show validation methods using “tolerance triangle”.

FIG. 1 shows a state-of-the-art blood pressure measuring system 102 which uses the following control techniques for continuous non-invasive CNAP methods to determine the blood pressure signal p_{A}(t): A plethysmographic system 103, 104, for example in the form of a finger cuff 105, is attached to an extremity 101 or a body part of a living being in which an artery A is located—such as the finger 101, a wrist or temple—and thus illuminated by means of a light source 103. The light that flows through this extremity 101 or is reflected by the bone lying in the extremity (e.g. carpal, temple) is registered by a suitable light detector 104 and is an inverse measure of the arterial blood volume in the extremity (plethysmographic signal v(t)). The more blood there is in the extremity, the more light is absorbed and the smaller the plethysmographic signal v(t).

The signal v(t) is now fed to a control mechanism 106 and a control value u(t) is determined, which subsequently changes the contact pressure p_{C}(t) at the extremity 101 generated by a pressure generating unit 108. The contact pressure p_{C}(t) acts at that point where the plethysmographic signal v(t) is also determined. The condition of the control mechanism determines that v(t) or certain frequency contents of v(t) (referred to as v_{F}(t)) are kept constant by the applied contact pressure p_{C}(t). If v(t) thus wants to change, then the control mechanism 106 adjusts the pressure p_{C}(t) via the control value u(t) in such a way that v(t) remains constant.

If this control condition is met—i.e. v(t) and thus the blood volume in the extremity 101 remains constant over time—the pressure difference between the intra-arterial pressure p_{A}(t) and the external contact pressure p_{C}(t)—the so-called transmural pressure p_{T}(t)—is zero. Thus the contact pressure p_{C}(t) corresponds to the intra-arterial pressure p_{A}(t) in the extremity. This contact pressure p_{C}(t) provided by the pressure generation unit 108 can be measured by means of a pressure sensor or manometer.

An essential component, namely the comparator of the controlled system is located inside the extremity (e.g. fingers). The so-called comparator is the comparison element in which the “reference variable” p_{A}(t) is compared with the “controlled variable” p_{C}(t). If p_{A}(t) changes compared to p_{C}(t), volume fluctuations occur which are recorded via the plethysmographic signal v(t).

The control loop according to FIG. 1 therefore only functions if the human comparator—i.e. the extremity 101 or the finger—is also introduced into the CNAP system. This human comparator in the form of a test person—or more precisely, the comparator function—had to be present in the past when the devices were tested or validated. The current blood pressure in the artery of the test person p_{A}(t) is thus compared with the contact pressure p_{C}(t) and the basic comparator function

v(t)=f(p_{C}(t)−p_{A}(t)) (1)

is generated. The current blood pressure in the artery p_{A}(t) is also not directly available to the CNAP system, which is the advantage of this non-invasive CNAP system. However, in comparison to other test systems, such as for ECG or the test systems for intermittent blood pressure monitors mentioned above, it is not possible to simply introduce a biological signal at a measuring sensor and thus have the device under test generate a measured value. The biological signal available to a CNAP system is directly influenced and changed by the measuring method or the contact pressure p_{C}(t).

For the verification, testing and validation of CNAP systems, it is advantageous if the comparator function can be made available in a standardized way, which is described in the following using embodiment variants of validation methods and validation apparatuses.

FIG. 2 shows a block diagram of the validation apparatus according to the invention. Instead of the extremity or finger, a simple finger dummy 201 is in contact with the plethysmographic system 203, 204. The plethysmographic signal v(t) is generated by the light source 203 and a light sensor 204. The signal v(t) is fed to the control mechanism 206 and a control value u(t) is determined, which subsequently changes a contact pressure p_{C}(t).

In this embodiment variant, care is taken to ensure that the same CNAP components are active as those used in accordance with the state of the art in FIG. 1. This is necessary for the validation proof described later. Ideally, the CNAP devices are designed in such a way that they are put into simulator mode and can thus virtually validate themselves. Thus the components of the blood pressure monitor 202 in FIG. 2 correspond to those of the blood pressure monitor 102 in FIG. 1; the light source 203 corresponds to the light source 103, etc. The supplementary system components are explained as follows in accordance with FIG. 2:

The finger dummy 201 is primarily responsible for ensuring that the contact pressure p_{C}(t) provided by the pressure generation unit 208 can actually occur in the blood pressure measuring system 202 by the contact pressure p_{C}(t) meeting a counterpart. The contact pressure p_{C}(t) is often generated by means of an inflatable finger cuff 205, this cuff could even burst if there is no counterpart.

Furthermore, the finger dummy 201 should have similar haptic properties as an actual finger. Especially the viscosity properties shall be simulated, e.g. by using a gel or gelatine as material for the finger dummy 201, which is surrounded by an elastic skin. This is advantageous for simulating the coupling of the light source 203 and light sensors 204.

The finger dummy 201 preferably has similar optical characteristics as an actual finger of a living being. For example, the absorption constant of the finger dummy 201 is chosen for the light wavelength occurring in the light source 203 as it occurs in a finger that is pressed down with a contact pressure p_{C}(t) above the systolic blood pressure. The amount of light that is transmitted through a finger that is squeezed via the systolic blood pressure should at least also be able to be transmitted through the finger dummy 201, but not significantly more.

The finger dummy 201 thus meets minimum haptic and optical requirements; however, in contrast to an actual finger, it does not have to modulate the light signal in order to bring the information about the arterial blood pressure curve p_{A}(t) into the CNAP system.

According to the invention, this is supposed to work by modulating the light source 203. In the measurement phase as shown in FIG. 1, light source 103 is always lit at constant light energy. It shall now be possible to change or modulate this light energy in a validation or test phase via a coupling interface 209. The light modulated in this way now flows through the finger dummy 201 and hits the light sensor 204, which receives the light regardless of whether the light modulations are caused by changes in the absorption in a finger or by modulations of the light intensity in the light source 203. The resulting signal v(t) is then fed to the control mechanism 206, which determines a control value u(t), and the contact pressure p_{C}(t) is changed, which in turn acts on the finger dummy 201.

Now the comparison element or comparator required for each control system is still missing, in which the reference variable p_{A}(t) is compared with the controlled variable p_{C}(t). A simulation module 210 is provided for this purpose, which receives as input parameters the contact pressure p_{C}(t) of the blood pressure measuring system 202 on the one hand, and a blood pressure curve p_{A}(t) previously recorded on a storage medium 211 on the other hand. The simulation module 210 calculates the plethysmographic signal v(t) according to equation (1)—the so-called comparator function of the simulation module 210—and feeds it to the coupling interface 209 to ensure the modulation of the light in the light source 203.

FIGS. 3a and 3b show the changes in the control of light source 103 (FIG. 1: Measurement phase or state of the art) and 203 (FIG. 2: Validation phase of the invention), respectively, wherein LEDs are preferably used as light sources 103, 203. It is known that the signal yield of an LED is better if the LED is controlled with electronic pulses. It is possible to generate higher LED currents for a short time, although the total energy consumption of the LED is reduced. The information about the switch-on times of the pulses is transmitted to the light receiver 104, 204. FIG. 3a shows such electronic control pulses during the measuring phase. There are only control pulses which either switch the light source 103 on completely (state 1) or switch it off (state 0).

FIG. 3b now shows how the energy of the switch-on pulses can be modulated. In order for the complete information of a blood pressure curve to be modulated and thus transmitted to the CNAP system via the light source 203, the height of the electronic pulse should be equipped with a minimum of 12 bit coding up to a maximum of approx. 16 bit coding. According to the invention, all other encodings are also possible.

FIGS. 4a and 4b show a typical pressure-volume diagram (p-v diagram). This information is necessary to determine the function from equation (1) and thus describe the comparator function of simulation module 210 in further detail below. The p-v diagram is usually determined in the open-loop phase, i.e. at the beginning of each measurement, where the “oscillometric principle” or the “principle of maximum amplitude” is used to search for the maximum pulsations and thus for the mean arterial blood pressure mBP.

FIG. 4a shows that at the point:

v(t)=maximum pulsations/contact pressure p_{Co}(t)=mBP

the operating point (setpoint) was found. For this purpose, the contact pressure p_{C}(t)—in this example using a ramp function—was changed and the corresponding plethysmographic signal v(t) was determined. It can be seen that v(t) has a pulsatile component as well as a kind of “constant component”. The pulsatile component is caused by the arterial blood pressure fluctuations and its frequency naturally corresponds to the heart rate. The so-called constant component, on the other hand, is caused by the absorption of light by bones, skin, tissue, venous blood, interstitial fluid and also by the degree of filling of the artery or arteries. The venous blood and the interstitial fluid can be displaced by the contact pressure p_{C}(t), therefore the constant component of these components changes with the contact pressure p_{C}(t). The constant component also changes significantly with the compression of the artery or arteries in the extremity, such as the finger.

To determine the pulsatile component from v(t), the signal is filtered with a high-pass filter. Mathematically speaking, this process corresponds to the determination of the first derivative after time. As the pressure p_{C}(t) increases, the amplitudes of the pulses first increase, only to become smaller again from a certain pressure—as described above from the mean arterial blood pressure mBP or at the point p_{Co}(t). The amplitudes of the pulses behave according to a bell curve. A typical picture of the “oscillometric envelope”, as is also known from automatic intermittent blood pressure monitors, emerges.

The constant component is smallest at low contact pressure p_{C}(t), because the light is absorbed not only by bones, skin and tissue, venous blood and interstitial fluid, but also to a large extent by arterial blood, which—averaged over the pulsations—is located in the artery. With increasing pressure, the constant component also increases and at a contact pressure p_{C}(t) far above the systolic blood pressure sBP, the arterial blood is completely displaced from the artery and saturation occurs. The light is only absorbed by bone, skin and tissue. The constant component reaches a maximum.

FIG. 4b shows the same p-v diagram in more detail. The constant component of v(t) has been determined and you can see that it corresponds to an S-curve. This S-curve shaped constant component can also be determined by mathematically integrating the bell-shaped “oscillometric envelope”. The maximum slope of the S-curve cₘₐₓ corresponds to the maximum amplitude or height of the bell curve.

The comparator function sought for the finger essentially corresponds to the S-curve shape of the constant component. The first derivative of this comparator function also corresponds to the oscillometric bell curve from the distribution of the pulse amplitudes. As shown in FIG. 4b, the extreme values Vₘᵢₙ (constant component if p_{C}(t)=0) and Vmax (constant component if p_{C}(t)>>sBP) and the operating point at V₀/p_{Co}(t)=mBP can be used to parameterize the S-curve in a present embodiment variant of the comparator function. The gradient at the point of the operating point corresponds to cₘₐₓ according to FIG. 4b.

The following system of equations can be set up for the comparator function:

$$v ( t ) = \{ \begin{matrix}\begin{matrix}{V}_{min} + ( {V}_{0} - {V}_{min} ) \cdot \\ {e}^{\frac{{c}_{max}}{{V}_{0} - {V}_{min}} ( {p}_{C} ( t ) - {p}_{A} ( t ) )}\end{matrix} & ∀ {p}_{C} ( t ) < {p}_{A} ( t ) \\ \begin{matrix}{V}_{max} - ( {V}_{max} - {V}_{0} ) \cdot \\ {e}^{- \frac{{c}_{max}}{{V}_{0} - {V}_{min}} ( {p}_{C} ( t ) - {p}_{A} ( t ) )}\end{matrix} & ∀ {p}_{C} ( t ) > {p}_{A} ( t )\end{matrix} \qquad ( 2 )$$

In this case, the slope k behaves as follows:

$$k ( t ) = \frac{dv}{dp} = \{ \begin{matrix}{c}_{max} \cdot {e}^{\frac{{c}_{max}}{{V}_{0} - {V}_{min}} ( {p}_{C} ( t ) - {p}_{A} ( t ) )} & ∀ {p}_{C} ( t ) < {p}_{A} ( t ) \\ {c}_{max} \cdot {e}^{- \frac{{c}_{max}}{{V}_{0} - {V}_{min}} ( {p}_{C} ( t ) - {p}_{A} ( t ) )} & ∀ {p}_{C} ( t ) > {p}_{A} ( t )\end{matrix} \qquad ( 3 )$$

According to the invention, other S-curve-shaped functions can also be used, such as the Areasinus hyperbolicus (arsinh) or the Gaussian distribution or sum function Φ, which can be applied in suitable variants of these functions.

In further embodiment variants, the comparator function can be determined from other mathematical methods. For example, the measurement of the v(t) amplitudes at different contact pressures p_{C}(t) is useful, wherein these v(t) amplitudes can then serve as support points for any kind of empirical function. The different v(t) amplitudes at different contact pressures p_{C}(t) can be determined in an open loop phase at the beginning of the measurement.

Experiments have shown that it is also advantageous to map other physiological properties of the comparator function. As described above, the constant component of v(t) is also dependent on the venous blood and the interstitial fluid, because both components are also dependent on the contact pressure p_{C}(t). The proportion of venous blood disappears relatively quickly when the contact pressure rises above the venous blood pressure—i.e. to p_{C}(t)>>20 mmHg—where the operating pressure of CNAP devices usually lies.

The case is different with the proportion of interstitial fluid. Firstly, pulsatile changes in p_{C}(t) cause a constant squeezing out and sucking in of interstitial fluid into the measuring finger. This has such an effect on v(t) that a hysteresis is formed.

This hysteresis can be represented mathematically as follows:

$${v}_{hys} ( t ) = v ( t ) - {c}_{hys} \cdot k ( t ) \cdot \frac{d ( {p}_{C} ( t ) - {p}_{A} ( t ) )}{dt} \qquad ( 4 )$$

where vhys(t) is the plethysmographic signal subject to hysteresis and c_{hys} is a constant characteristic of the hysteresis. v(t) is the plethysmographic signal determined from one of the methods described above; k(t) is the first derivative dv(t)/dt.

Secondly, experience shows that it takes up to five minutes for the interstitial fluid to be pressed out of the finger until a stable equilibrium is achieved. As long as the constant component drifts due to the interstitial liquid component, a kind of parallel shift of the S-curve comparator function occurs. Such “fluid shifts” can also occur again and again during operation, e.g. by repositioning the measuring finger or after the patient has taken in fluid by drinking, but also by infusions. It is therefore advantageous to re-measure the S-curve or its parameters at regular intervals. Vasomotor activity also influences the S-curve comparator function, which makes it useful to check and, if necessary, correct it.

For checking and, optionally, correcting the comparator function during operation, it is conceivable to determine v(t) amplitudes at different contact pressures p_{C}(t) in a short open-loop phase, but also evaluation sequences during the closed-loop phase.

If the S-curve comparator function is present and this is also checked regularly and optionally corrected according to an embodiment variant, the control loop of the present simulator can be closed as described in FIG. 2. This control loop is summarized as follows: an arterial blood pressure curve p_{A}(t) is stored on a storage medium 211. This is read out and fed to simulation module 210 in the correct time sequence, in which the S-curve comparator function is mapped. Furthermore, the contact pressure p_{C}(t) is an input variable of the simulation module 210. The output signal of the simulation module 210 is the plethysmographic signal v(t), with which the light source 203 is now modulated. The light flows through the finger dummy 201 and hits the light sensor 204. Said light sensor 204 cannot distinguish whether the signal v(t) arriving at it was modulated by absorption in a finger or by the simulation module. The signal v(t) is fed to the control mechanism 206, which determines the manipulated variable u(t) and subsequently changes the contact pressure p_{C}(t)—which in turn acts on the finger dummy 201. The signal p_{C}(t) is also fed to the simulation module 210, where it is again compared with the arterial blood pressure curve p_{A}(t) on the storage medium 211.

Nota bene: the simulation module 210 of these embodiment variants generates analog signals: both the light is modulated and thus generates a signal v(t), and the contact pressure p_{C}(t) in the blood pressure measuring system 202, for example in the finger cuff 205, is also effective.

In other embodiment variants, the control loop can also be digitally reproduced on a computer. In this case, certain electronic elements such as the plethysmographic system 203, 204 or the means for changing the contact pressure p_{C}(t) must be reproduced digitally. This has the advantage that other important digital elements such as the control mechanism 206, which is mainly in the form of an algorithm in software codes, can be easily tested on a computer.

FIGS. 5a to 5d describe possible methods for validation. It must first be mentioned that the test systems and simulators described here can only be used efficiently for validation purposes if the functionality of the test systems and simulators can also be validated. It must be demonstrated that the systems function with sufficient accuracy.

For medical devices in general and for blood pressure monitors in particular, standards apply which have defined tolerance limits. These tolerance limits must be met by new devices compared to gold standard methods when they are introduced to the market. For example, intermittent blood pressure monitors have an average difference to the gold standard of 5 mmHg and a standard deviation of 8 mmHg. A separate standard for continuous blood pressure monitors is currently being developed.

In order to prove that the test system at hand also complies with this standard and the tolerance limit, the following procedure could be applied: During the recording of a blood pressure curve, which can later also be imported into a CNAP simulator system, the true intra-arterial blood pressure (IBP) should be recorded simultaneously by means of a catheter (gold standard) and a non-invasive blood pressure curve (CNAP). Ideally, blood pressure curves should not only be recorded for one patient, but for a certain number of patients, as specified in the standard. From the statistical evaluation, a deviation between CNAP and the gold-standard IBP is obtained, which ideally lies within the prescribed tolerance limit.

Exactly this CNAP system used for recording, including all associated components, is now being converted into a simulator. The comparator function of the respective patient is imported into simulation module 210 and with the corresponding IBP the first simulator recording is generated, designated in FIGS. 5a-d as “1^{st} SIMU”. This is done for all patient files and a data set of 1^{st} SIMUs is created. This dataset can now be compared with both the IBP dataset and the CNAP dataset. The sum of the datasets to each other must—as shown in FIG. 5a—be within a tolerance triangle with the side length “Max Limit”, wherein “Max Limit” describes the tolerance limit prescribed in a standard. FIG. 5b shows an illustration for an acceptable, validated simulator system that is within the tolerance triangle. All three data sets—IBP (gold standard), CNAP and 1^{st} SIMU—have differences between them that are within the tolerance limits. This is not the case in FIG. 5c, where, for example, the difference between IBP and 1^{st} SIMU is too large.

Although the exact same CNAP components (sensors, light system, control system, contact pressure device, etc.) are used for the first simulations, the 1^{st} SIMU blood pressure curve will not be completely equivalent to the CNAP curve. This is because the comparator function of the simulation module 210 was of course only simulated. The better the CNAP and 1^{st} SIMU correlate, the more accurate the S-curve comparator function was simulated.

However, the test system is not built to subsequently test the same CNAP components. FIG. 5d now shows how new CNAP systems or subsystems can be tested and, above all, which tolerance limits must be observed for validations. If a new data set with new CNAP components is created by importing IBP as the gold standard into the simulator with the new CNAP components, the new data system must have differences within the tolerance limits for all three data sets—IBP (gold standard), CNAP and 1^{st} SIMU. In other words, the new SIMU data set must lie within a tetrahedron which, according to FIG. 5d, spans the “tolerance triangle” with the side length “Max. Limit” known from FIG. 5a.

1-15. (canceled)

16. A method for validating a continuously measuring, non-invasive blood pressure measuring system equipped with a plethysmographic system which is suitable for obtaining, in a measuring phase, a plethysmographic signal v(t) at an extremity, the method comprising:
feeding the signal v(t) from the plethysmographic system to a control mechanism which changes the contact pressure p_{C}(t) of the blood pressure measuring system at the extremity via a control value u(t);
based on the resulting contact pressure p_{C}(t), continuously determining the arterial blood pressure curve p_{A}(t); and
in a validation or test phase, feeding a signal derived from a previously recorded blood pressure curve into the blood pressure measuring system via a coupling interface.

17. The method according to claim 16, wherein an output signal is fed to the coupling interface from a simulation module, to which a blood pressure curve recorded on a storage medium and the contact pressure p_{C}(t) of the blood pressure measuring system are fed as input signals, wherein a simulated, continuous blood pressure curve is determined by the blood pressure measuring system.

18. The method according to claim 17, wherein the simulated, continuous blood pressure curve is compared with the blood pressure curve coupled from the storage medium into the simulation module and differences between the stored and the simulated blood pressure curve are used as a criterion for validating the blood pressure measuring system.

19. The method according to claim 16, wherein the previously recorded blood pressure curve for the validation of the blood pressure measuring system is obtained from an intra-arterial blood pressure measurement or a non-invasive blood pressure measurement of a preceding clinical measurement.

20. The method according to claim 16, wherein
at the same time as a non-invasive blood pressure measurement in a living being, an intra-arterial blood pressure measurement is performed and recorded in the living being;
the recorded intra-arterial blood pressure curve or the recorded non-invasive blood pressure curve are coupled into the blood pressure measuring system and blood pressure curves simulated by the blood pressure measuring system are generated; and
the simulated blood pressure curves are used as a criterion for validating the blood pressure measurement system.

21. The method according to claim 16, wherein the light intensity of at least one light source of the plethysmographic system is modulated, in the validation or test phase, based on the previously recorded blood pressure curve.

22. The method according to claim 16, wherein electronic elements of the blood pressure measuring system, such as the plethysmographic system, or means for changing the contact pressure p_{C}(t) system, in the validation or test phase, are simulated as a digital software model.

23. The method according to claim 17, wherein an S-shaped or bell-curve comparator function is calculated in the simulation module.

24. The method according to claim 23, wherein the S-shaped or bell-curve comparator function is determined in an open-loop phase of the blood pressure measuring system before the start of data recording for blood pressure measurement.

25. The method according to claim 24, wherein the S-shaped or bell-curve comparator function is checked and, if necessary, corrected during the data recording of the blood pressure measurement.

26. An apparatus for validating a continuously measuring, non-invasive blood pressure measuring system comprising a plethysmographic system capable of obtaining, in a measuring phase, a plethysmographic signal v(t) at an extremity, the apparatus comprising:
a control mechanism to which the signal v(t) from the plethysmographic system is fed, which changes the contact pressure p_{C}(t) at the extremity via a manipulated value u(t); and
an evaluation unit which continuously determines the arterial blood pressure curve p_{A}(t) based on the resulting contact pressure p_{C}(t);
wherein the blood pressure measuring system has a coupling interface via which, in a validation or test phase, a signal derived from a previously recorded blood pressure curve can be input into the blood pressure measuring system.

27. The apparatus according to claim 26, wherein the plethysmographic system comprises at least one light source and at least one light detector, and wherein the coupling interface is adapted to modulate the light intensity of the light source based on the signal of the recorded blood pressure curve.

28. The apparatus according to claim 26, wherein the blood pressure measuring system comprises a simulation module which is connected on the input side on the one hand to a storage medium which provides a signal of the previously recorded blood pressure curve p_{A}(t) and on the other hand to a pressure generating device of the plethysmographic system, which provides a signal of the contact pressure p_{C}(t), wherein the simulation module is suitable for calculating a comparator function based on the two input signals and transmitting it to the coupling interface.

29. The apparatus according to claim 26, wherein the device comprises a finger dummy which can be brought into contact with the plethysmographic system in the validation or test phase of the blood pressure measuring system instead of the extremity in the measuring phase.

30. The apparatus according to claim 29, wherein the haptic or optical properties of the finger dummy correspond to the extremity of a living being.

31. The apparatus according to claim 26, wherein the light source is an LED and the light detector is a photodiode.
